(19) United States
(12) Patent Application Publication
(10) Pub. No.: US 2024/0139133 A1
(43) Pub. Date: May 2, 2024

(54) TOPICAL NAPROXEN FORMULATIONS AND THEIR USE

(71) Applicant: SMARTECH TOPICAL, INC., Santee, CA (US)

(72) Inventor: Thomas HNAT, San Diego, CA (US)

(73) Assignee: SMARTECH TOPICAL, INC., Santee, CA (US)

(21) Appl. No.: 18/559,019

(22) PCT Filed: May 5, 2022
(86) PCT No.: PCT/US2022/027935

§ 371 Date: Nov. 3, 2023

Related U.S. Application Data
(60) Provisional application No. 63/184,631, filed on May 5, 2021.

(51) Int. Cl. A61K 31/192; A61K 9/00; A61K 31/00; A61K 31/167; A61K 31/593; A61K 47/10; A61K 47/14; A61K 47/20; A61K 47/22; A61K 47/38

(52) U.S. Cl. CPC A61K 31/192; A61K 9/0014; A61K 31/167; A61K 31/593; A61K 31/658; A61K 47/10; A61K 47/14; A61K 47/20; A61K 47/22; A61K 47/38

(57) ABSTRACT

Topical naproxen formulations comprising long chain monounsaturated fatty acids, long chain monounsaturated fatty alcohols, terpenes, or combinations thereof and a solvent mixture comprising ethanol, propylene glycol, 2-(2-Ethoxyethoxy)ethanol, and optionally dimethylsulfoxide (“DMSO”), wherein the formulation comprises about 5.0 wt % or less water and is preferably anhydrous.

The present application claims the benefit of U.S. Provisional Application No. 63/184,631, filed May 5, 2021, from which priority is claimed and which is hereby incorporated by reference in its entirety including all tables, figures and claims.

### BACKGROUND OF THE DISCLOSURE

The following discussion of the background of the disclosure is merely provided to aid the reader in understanding the disclosure and is not admitted to describe or constitute prior art to the present disclosure.

Cyclooxygenase (COX, also known as prostaglandin-endoperoxide synthase), refers to a family of enzymes responsible for formation of prostanoids, including thromboxane and prostaglandins such as prostacyclin, from arachidonic acid. As the prostanoids are mediators of pain and inflammation, COX represents a common pharmaceutical target. Agents that inhibit prostaglandin G/H synthase (cyclooxygenase or COX), an enzyme that catalyzes the production of prostanoids, including prostaglandins, prostacyclin and thromboxane, from arachidonic acid, are referred to as COX inhibitors. Common nonsteroidal anti-inflammatory drugs (NSAIDs), such as aspirin and ibuprofen, exert their effects through inhibition of enzymes COX-1 and COX-2, while NSAIDS such as celecoxib and etoricoxib are specific to the COX-2 isozyme. Acetaminophen, while not considered an NSAID because it has only minor anti-inflammatory activity, treats pain by blocking COX-2 while also inhibiting endocannabinoid reuptake.

The use of COX inhibitors in a topical formulation may be beneficial in reducing the likelihood of a patient experiencing adverse effects associated with systemic therapy. Medications applied directly to the skin may be either intended for local action or systemic effects. Topically applied medications (e.g., topical patches, creams, gels, ointments, solutions, etc.) may be intended to reach local tissue to achieve the desired therapeutic effect, or may act transdermally to result in systemic concentrations comparable with orally administered medications.

There are several topical NSAID products available in the United States approved to treat painful conditions. Diclofenac sodium 1% gel (Voltaren Gel) is approved for the relief of pain due to osteoarthritis in joints amenable to topical treatment, such as the knees and those of the hands. This product contains a variety of additional ingredients in the vehicle including isopropyl alcohol, propylene glycol, and water to assist in drug penetration of the skin. Diclofenac sodium topical solution 1.5% w/w (PENNSAID) is indicated for the treatment of signs and symptoms of osteoarthritis of the knee(s). Additional absorption-enhancing ingredients in this product include DMSO, propylene glycol, water and alcohol. A diclofenac epolamine 1.3% topical patch (Flector Patch) is indicated for the topical treatment of acute pain due to minor strains, sprains, and contusions. The patch is composed of an adhesive material containing 1.3% diclofenac epolamine, applied to a non-woven polyester felt backing and covered with a polypropylene film release liner which is removed prior to application.

Evidence indicates that topical formulations can achieve therapeutic concentrations of drug in localized tissue while maintaining low serum levels of drug and potentially avoiding systemic toxicity. Topical diclofenac preparations have a reported maximum serum concentration that is 0.4-2.2% of the maximum serum concentration achieved with oral diclofenac, resulting in significantly lower systemic exposure. High drug concentration at the site of action paired with low systemic concentrations can lead to efficacy greater than or equal to that of systemic NSAIDs with a reduced risk of adverse effects.

### BRIEF SUMMARY OF THE DISCLOSURE

In a first aspect, the present disclosure provides topical Naproxen formulations. These topical formulations comprise or consist of:
between about 0.5% wt % and about 25% wt % Naproxen;
between about 1.0 wt % and about 15.0 wt % of long chain monounsaturated fatty acids, long chain monounsaturated fatty alcohols, terpenes, or combinations thereof;
between 0 and about 5.0 wt % of a poloxamer;
between 0 and about 5.0 wt % of a pharmaceutically acceptable cellulosic excipient;
between 0 and about 5.0 wt % of α-tocopheryl polyethylene glycol succinate (“TPGS” or “Vitamin E TPGS”);
a solvent mixture comprising ethanol, propylene glycol, 2-(2-Ethoxyethoxy)ethanol, and optionally dimethylsulfoxide (“DMSO”); and
wherein the formulation comprises about 5.0 wt % or less water, preferably about 1.0% or less, and most preferably 0% water.

In certain embodiments, the topical formulations comprise or consist of:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between 0 and about 50 wt % 2-(2-Ethoxyethoxy)ethanol;
between about 1 wt % and about 5 wt % TPGS;
between 0 and about 50 wt % Poloxamer 188;
between 0 and about 5 wt % lidocaine;
between 0 and about 5 wt % cannabidiol;
between 0 and about 0.1 wt % vitamin D3;
ethanol q.s. to 100 wt %; and
wherein the formulation is anhydrous as that term is defined below.

In certain embodiments, the topical formulations comprise or consist of:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
between about 1 wt % and about 4 wt % TPGS;
between 0 and about 50 wt % Poloxamer 188;
between 0 and about 5 wt % lidocaine;
ethanol q.s. to 100 wt %; and
wherein the formulation is anhydrous as that term is defined below.

In certain embodiments, the topical formulations comprise or consist of:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
between about 1 wt % and about 4 wt % TPGS;
ethanol q.s. to 100 wt %; and
wherein the formulation is anhydrous as that term is defined below.

In certain embodiments, the topical formulations comprise or consist of:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
ethanol q.s. to 100 wt %; and
wherein the formulation is anhydrous as that term is defined below.

The naproxen may be present in the topical formulations of the present invention as a free acid or as various salts (e.g., naproxen sodium) or esters (e.g., naproxen methyl ester).

In preferred embodiments, the topical formulations of the present invention are clear, transparent, and slightly viscous. In these formulations, Naproxen does not form co-crystals with any of the components of the formulation, but rather is solubilized in the formulation.

In various embodiments, the topical formulation further comprises one or more COX inhibitors selected from the group consisting of cannabinoids (e.g., tetrahydrocannabinol (D9-THC), tetrahydro-cannabinolic acid-A (THCA-A), cannabidiol (CBD), cannabidiolic acid (CBDA), cannabigerol (CBG) and cannabigerolic acid (CBGA)), Acetaminophen, Benzydamine, Bufexamac, Diclofenac, Etofenamate, Flufenamic acid, Ibuprofen, Indomethacin, Ketoprofen, and salicylates (e.g., salicylic acid, salicin, diflunisal, magnesium salicylate, choline salicylate).

In certain embodiments, the topical formulation provides a percutaneous absorption of Naproxen of at least 7% of the amount present in the formulation. Percutaneous absorption (or skin permeation) can be visualized as consisting of a series of steps in sequence: sorption of a penetrant molecule onto the surface layers of stratum corneum, diffusion through it and the viable epidermis. At the papillary layer of the dermis, the molecule is taken up into the microcirculation for subsequent systemic distribution. Methods for measuring percutaneous absorption of topically applied drugs are known in the art. See, e.g., Kezic, Hum. Exp. Toxicol. 2008 27(4): 289-95. doi: 10.1177/0960327107085825. A formulation according the present claims preferably provides a percutaneous absorption of Naproxen of at least 10%.

In most preferred embodiments, the topical formulation comprises no water; that is, the formulation is anhydrous. By “wt % water”, “no water” or “anhydrous” is meant that the formulation contains the indicated weight percentage of water or does not include the use of water, either added as water per se or as a component of one of the liquid solvents. By way of example, 95% ethanol, which is an azeotrope comprising 5% water, is not used in an anhydrous formulation as there would be water in the ethanol. Water that is a component of a hydrated ionic compound (a compound that is a “hydrate” containing water of hydration) or that results from hygroscopic absorption, however, may be present in such an anhydrous formulation.

The term “wt %” as used herein refers to (mass of the component/total mass of the formulation)×100. By way of example, 2 wt % naproxen refers to 2 g of naproxen per 100 g of the formulation.

The term “long chain monounsaturated fatty acid” refers to fatty acids having at least 14 carbons and a single double bond. The term “long chain monounsaturated fatty alcohol” refers to an equivalent alcohol (that is, an —OH group attaches to the terminal carbon rather than an alkoxy). For example, the formula for oleic acid is CH₃(CH₂)₇CH═CH(CH₂)₇COOH, while the formulation for the equivalent oleyl alcohol is CH₃(CH₂)₇—CH═CH—(CH₂)₈OH. Examples of monounsaturated fatty acids falling within this group include, but are not limited to, the following:
Myristoleic acid 14:1 (n-5)
Palmitoleic acid 16:1 (n-7)
cis-Vaccenic acid 18:1 (n-7)
Vaccenic acid 18:1 (n-7)
Paullinic acid 20:1 (n-7)
Oleic acid 18:1 (n-9)
Elaidic acid (trans-oleic acid) 18:1 (n-9)
11-Eicosenoic acid (gondoic acid) 20:1 (n-9)
Erucic acid 22:1 (n-9)
Brassidic acid 22:1 (n-9)
Nervonic acid 24:1 (n-9)
Sapienic acid 16:1 (n-10)
Gadoleic acid 20:1 (n-11)
Petroselinic acid 18:1 (n-12)

In various embodiments, the long chain monounsaturated fatty acids and/or long chain monounsaturated fatty alcohols present in the formulation are C16:1 to C22:1 fatty acids or alcohols. In preferred embodiment, the long chain monounsaturated fatty acids present in the formulation comprise or consist of between about 1 and about 15 wt % oleic acid, oleyl alcohol, or a mixture thereof, more preferably between about 1 and about 10 wt % oleic acid, oleyl alcohol, or a mixture thereof, and most preferably between about 1 and about 5 wt % oleic acid, oleyl alcohol, or a mixture thereof. In certain embodiments, the long chain monounsaturated fatty acids present in the formulation comprise or consist of about 1 wt % oleic acid, oleyl alcohol, or a mixture thereof, about 2 wt % oleic acid, oleyl alcohol, or a mixture thereof, about 3 wt % oleic acid, oleyl alcohol, or a mixture thereof, about 4 wt % oleic acid, oleyl alcohol, or a mixture thereof, about 5 wt % oleic acid, oleyl alcohol, or a mixture thereof, about 6 wt % oleic acid, oleyl alcohol, or a mixture thereof, about 7 wt % oleic acid, oleyl alcohol, or a mixture thereof, about 8 wt % oleic acid, oleyl alcohol, or a mixture thereof, about 9 wt % oleic acid, oleyl alcohol, or a mixture thereof, or about 10 wt % oleic acid, oleyl alcohol, or a mixture thereof.

Poloxamers are nonionic triblock copolymers composed of a central hydrophobic chain of polyoxypropylene flanked by two hydrophilic chains of polyoxyethylene. these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content. Examples of poloxamers which may find use in the present disclosure include, but are not limited to, poloxamer-101, -105, -105 benzoate, -108, -122, -123, -124, -181, -182, -182 dibenzoate, -183, -184, -185, -188, -212, -215, -217, -231, -234, -235, -237, -238, -282, -284, -288, -331, -333, -334, -335, -338, -401, -402, -403, and -407. In preferred embodiment, the poloxamer present in the formulation comprises, or consists of, between about 0.1 and about 5 wt % poloxamer-188 or contains no poloxamer

Cellulose and its derivatives (e.g., ether and ester derivatives) are among the excipients frequently used in pharmaceutical compounded and industrialized products with various purposes. Among their uses are as suspending agents in oral liquid preparations and as viscosity increasing agents in topical formulations. Examples of pharmaceutically acceptable cellulosic excipients which can find use in the disclosure include, but are not limited to, hydroxypropylcellulose, hydroxypropyl methylcellulose, carboxymethylcellulose, methylycellulose, ethylcellulose, hydroxyethyl cellulose, hydroxyethylmethylcellulose and ethyl hydroxyethylcellulose. In preferred embodiment, the pharmaceutically acceptable cellulosic excipients present in the formulation comprises, or consists of, between about 1.0 and about 5 wt % of hydroxypropylcellulose. In certain embodiments, the cellulosic excipients in the formulation comprise or consist of about 1 wt % of hydroxypropylcellulose, about 2 wt % of hydroxypropylcellulose, about 3 wt % of hydroxypropylcellulose, about 4 wt % of hydroxypropylcellulose, or about 5 wt % of hydroxypropylcellulose. In certain other embodiments, the formulation is free of cellulosic excipients.

In certain embodiments, the topical formulations of the present invention comprise one or more anesthetic agents, e.g., procaine, chloroprocaine, tetracaine, cocaine, and benzocaine, dibucaine, lidocaine, mepivacaine, prilocaine, bupivacaine, levobupivacaine, ropivacaine, articaine, and etidocaine. Suitable amounts of such agents are up to about 5% of the formulation.

In certain embodiments, the topical formulations of the present invention are transparent. The term “transparent” as used herein refers to a formulation having the property of transmitting visible light without appreciable scattering so that bodies lying beyond the formulation are visible to the human eye. The visible spectrum in humans extends from about 380 to about 750 nm. While water at 20° C. exhibits a significant absorption at 860 nm, it is considered transparent for purposes of the present invention.

Most transparent media have refractive indices between 1 and 2 measured at the yellow doublet D-line of sodium, with a wavelength of 589 nanometers. By way of example, the refractive index of water is 1.33. In contrast, the refractive index of a turbid medium cannot be measured in transmission mode because of multiple light scattering effects, and is typically measured instead in reflection mode. Thus, in certain embodiments, the topical formulations of the present invention have a refractive index between 1 and 2 as measured using an Abbe refractometer in transmission mode at 20° C. at 589 nm.

In certain embodiments, the topical formulations of the present invention are slightly viscous. The term “slightly viscous” as used herein refers to a formulation having a viscosity of more than about 100 centipoise and less than about 5000 centipoise at 20° C. For comparison, the viscosity of water at 20° C. is 1 centipoise. In certain embodiments, a slightly viscous formulation is more than about 500 centipoise and less than about 2000 centipoise at 20° C.

The term “about” as used throughout the specification with regard to a value refers to no more than +/−10%. In certain embodiments, +/−10% of the given value may be replaced by +/−5% of the given value or +/−1% of the given value.

A list of exemplary formulations of the disclosure may be found in the following tables. In each case, the values recited in the tables can include +/−10% of each value within their scope, +1-5%, or +/−1%. In each case, the formulation is preferably anhydrous and forms a transparent, slightly viscous gel in which the naproxen is not in the form of a co-crystal, but rather is completely solubilized in the formulation. Most preferably, each formulation exhibits a refractive index between 1 and 2 as measured using an Abbe refractometer in transmission mode at 20° C. at 589 nm

| Ingredient | Wt % | Wt % | Wt % | Wt % |
|---|---|---|---|---|
| Oleic acid, oleyl alcohol, | 8.0 | 8.0 | 8.0 | 8.0 |
| or a combination thereof | | | | |
| DMSO | 20.0 | 20.0 | 20.0 | 20.0 |
| Ethanol | 33.0 | 32.0 | 31.0 | 30.0 |
| Naproxen | 1.0 | 2.0 | 3.0 | 4.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 8.0 | 8.0 | 8.0 | 8.0 |
| or a combination thereof | | | | |
| DMSO | 20.0 | 20.0 | 20.0 | 20.0 |
| Ethanol | 29.0 | 28.0 | 27.0 | 26.0 |
| Naproxen | 5.0 | 6.0 | 7.0 | 8.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 10.0 | 10.0 | 10.0 | 10.0 |
| or a combination thereof | | | | |
| DMSO | 20.0 | 20.0 | 20.0 | 20.0 |
| Ethanol | 23.0 | 22.0 | 21.0 | 20.0 |
| Naproxen | 9.0 | 10.0 | 11.0 | 12.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 20.0 | 20.0 | 20.0 | 20.0 |
| Ethanol | 17.0 | 16.0 | 15.0 | 14.0 |
| Naproxen | 13.0 | 14.0 | 15.0 | 16.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 13.0 | 13.0 | 13.0 |
| or a combination thereof | | | | |
| DMSO | 20.0 | 20.0 | 20.0 | 20.0 |
| Ethanol | 13.0 | 11.0 | 10.0 | 9.0 |
| Naproxen | 17.0 | 18.0 | 19.0 | 20.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 10.0 | 12.0 | 12.0 |
| or a combination thereof | | | | |
| DMSO | 20.0 | 20.0 | 20.0 | 20.0 |
| Ethanol | 15.0 | 15.0 | 9.0 | 20.0 |
| Naproxen | 15.0 | 15.0 | 15.0 | 10.0 |
| Lidocaine | 0 | 0 | 4.0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 2.0 | 2.0 | 0 |
| TOTAL | 100 | 100 | 100 | 100 |
| Ingredient | Wt % | Wt % | Wt % | Wt % |
| Oleic acid, oleyl alcohol, | 10.0 | 12.0 | 12.0 | 12.0 |
| or a combination thereof | | | | |
| DMSO | 20.0 | 20.0 | 20.0 | 20.0 |
| Ethanol | 20.0 | 12.0 | 21.0 | 17.0 |
| Naproxen | 10.0 | 10.0 | 5.0 | 5.0 |
| Lidocaine | 0 | 4.0 | 0 | 4.0 |
| Propylene Glycol | 10.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 2.0 | 2.0 | 2.0 |
| TOTAL | 100 | 100 | 100 | 100 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 45.0 | 45.0 | 0 | 0 |
| Ethanol | 18.0 | 13.0 | 18.0 | 9.0 |
| Naproxen | 10.0 | 15.0 | 10.0 | 15.0 |
| Lidocaine | 0 | 0 | 0 | 4.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 45.0 | 45.0 | 0 | 0 |
| Ethanol | 13.0 | 10.0 | 18.0 | 9.0 |
| Naproxen | 12.0 | 12.0 | 12.0 | 12.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 45.0 | 45.0 | 0 | 0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 45.0 | 45.0 | 0 | 0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 1.0 | 1.0 | 1.0 | 1.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 1.0 | 1.0 | 1.0 | 1.0 |
| TOTAL | 100 | 100 | 100 | 100 |

In a related aspect, the present disclosure provides methods for topically treating a pain episode at a location on the human body, comprising topically applying a topical Naproxen formulation according to the disclosure to the location. In various embodiments the pain episode is an acute pain episode or a chronic pain episode. Examples of pain episodes which may be treated include, but are not limited to, pain resulting from osteoarthritis, rheumatoid arthritis, mild-to-moderate inflammation and tissue injury, low back pain, inflammatory arthropathies (e.g., ankylosing spondylitis, psoriatic arthritis, reactive arthritis), tennis elbow, headache, postoperative pain, muscle stiffness and pain due to Parkinson's disease, and traumatic injury. In preferred embodiments, the present methods are for topically treating pain of osteoarthritis of the knee(s), comprising topically applying a topical Naproxen formulation according to the disclosure to the knee(s).

In certain embodiments, the dose of a Naproxen formulation according to the disclosure applied provides a Naproxen amount of about 80 mg, about 40 mg, about 30 mg, about 20 mg, or about 10 mg. In certain embodiments, for example, application of 4 mL of a 2 wt % Naproxen formulation will provide a topical dose of 80 mg of Naproxen; 2 mL will provide 40 mg, 1 mL will provide 20 mg, etc.

In some embodiments, a formulation of the disclosure is in the form of a gel, lotion, cream, spray, aerosol, ointment, emulsion, suspension, liposomal system, lacquer, patch, bandage, buccal tablet, wafer, sublingual tablet, suppository, vaginal dosage form or occlusive dressing. In a particular embodiment, the formulation is a gel. In some embodiments, a formulation of the present disclosure is applied directly to the skin as, for example, a gel, an ointment, or a cream or indirectly through a patch, bandage, or other occlusive dressing. A formulation of the disclosure may be applied once daily, or multiple times per day depending upon the condition of the patient. In some embodiments, said formulation is adapted for a once, twice, three times or four times daily administration for as long as desired, suitably on the order of days to weeks to months, or longer if desired. The compositions can be administered to any skin surface, including the hand, arms, trunk, back, legs, feet, etc.

It is to be understood that the disclosure is not limited in its application to the details of construction and to the arrangements of the components set forth in the following description or illustrated in the drawings. The disclosure is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

### DETAILED DESCRIPTION OF THE DISCLOSURE

The greatest hindrances in the topical, percutaneous delivery of COX inhibitors is the obstructive property of the stratum corneum (SC), the outermost layer of the skin, skin binding, skin metabolism, cutaneous toxicity and prolonged lag times. Different methodologies have been developed to enhance transdermal absorption, including the use of drug derivatives, super-saturated systems, physical approaches, and chemical penetration enhancers (sorption promoters) that facilitate the diffusion of drugs through the SC. In that regard, numerous chemicals have been used for their skin permeation promoting capacity, including fatty acids, fatty acid esters, fatty alcohols or fatty alcohol ethers, fatty ethers, lower alcohols, glycerol esters, polyhydric alcohols, diols, amides (e.g., N,N-diethyl-m-toluamide), amines, terpenes, polar solvents, pyrrolidones and derivatives thereof, sulfoxides, azone or laurocapram, surface active agents, lecithin, polyols, glycols, quaternary ammonium compounds, silicones, alkanoates, certain biologies, enzymes, complexing agents, macrocyclics, solvents, etc.

As used herein, “permeation enhancement” refers to increasing the permeability of the skin to an active pharmaceutical ingredient (API), so as to increase the rate at which the API permeates through the skin. Similarly, “permeation enhancer” (PE) refers to an agent or mixture of agents that achieve such permeation enhancement. A PE mixture suitable for the instant disclosure promotes penetration of an API through the skin by one or more of the following mechanisms: (1) by increasing the diffusivity of the drug in the skin; (2) by causing SC lipid-fluidization, which leads to decreased barrier function (a reversible action); (3) by increasing and optimizing the thermodynamic activity of the drug in the vehicle; (4) by affecting the partition coefficient of the drug; and (5) by increasing its release from the formulation into the upper layers of the skin.

In certain embodiments, a PE mixture suitable for the instant disclosure has one or more of the following characteristics: non-toxic, non-irritant, non-allergenic, and/or non-sensitizing to skin; pharmacologically inert, at least at the concentrations required to exert adequate permeation action; immediate, predictive, and/or reversible effect; easily incorporated into pharmaceutical preparations; and cosmetically acceptable.

Fatty Acid Permeation Enhancers

The PE mixture of the present disclosure preferably comprises one or more fatty acids such as a long chain fatty acids. For example, the fatty acid may be oleic acid (cis-9-octadecenoic acid), or a functional derivative thereof. In certain embodiments, the PE is a fatty acid ester, fatty alcohol or fatty alcohol ether, fatty ether, lower alcohol, glycerol ester, polyhydric alcohol, diol, amide (e.g., N,N-diethyl-m-toluamide), amine, terpene, polar solvent or a mixture thereof. In certain embodiments, the fatty acid is alkanoic acid, capric acid, diacid, ethyloctadecanoic acid, hexanoic acid, lactic acid, lauric acid, linoelaidic acid, linoleic acid, linolenic acid, neodecanoic acid, oleic acid (cis-9-octadecenoic acid), palmitic acid, pelargonic acid, propionic acid, or vaccenic acid. In certain embodiments, the PE is at least one of a C8-C22 fatty acid, such as isopropyl myristate.

While not wishing to be bound by any particular theory, the fatty acid PEs of the disclosure are believed to selectively perturb the intercellular lipid bilayers in the SC, thus enhancing the penetration of the SC by the API. In certain embodiments, differences in penetration enhancing effects may be adjusted by adjusting the number of double bonds and cis/trans configuration of the fatty acid isomers, based on the general trend that unsaturated fatty acids are more effective (e.g., more than 5-fold, 10-fold, 15-fold, 20-fold or more) in enhancing percutaneous absorption than their saturated counterparts, especially for lipophilic drugs/APIs.

In certain embodiments, the PE is oleic acid, linoleic acid, a-linolenic acid, arachidonic acid, palmitic acid, lauric acid, caprylic acid, iso stearic acid, isopropyl myristate, or myristic acid, optionally further comprising one or more of propylene glycol, ethanol, 2-ethyl-1,3-hexanediol, and dexpanthene. In certain embodiments, the PE is palmitic acid, and the topical formulation is formulated to enhance the penetration of an API to the SC (a particularly alkyl-rich region). In certain embodiments, the PE is myristic acid, and the topical formulation is formulated to enhance the penetration of an API to the epidermis. In certain embodiments, the PE is octyl salicylate, and the topical formulation is formulated to enhance the penetration of a water-soluble or oil-soluble API into the epidermis and dermis.

Additional fatty acid-based PEs can be found in MX 9705070, GR 1004995, US 2005-020552A1, WO 05/060540, CA 2,420,895, MX 9800545, WO 04/054552, NZ 537359, WO 98/18417, WO 96/30020, DE 4301783, U.S. Pat. Nos. 4,885,174, 4,983,396, NZ 222346, CA 1,280,974, and U.S. Pat. No. 4,626,539.

Terpene Permeation Enhancers

Due to their high enhancement effect and low skin irritation, terpenes can find use in pharmaceutical and cosmetic formulations as permeation enhancers. Terpenes, primarily extracted from medicinal plants, are volatile compounds with molecular components that are composed of only carbon, hydrogen and oxygen atoms. The basic chemical structure of terpenes consists of a number of repeated isoprene (C5H8) units which are used to classify terpenes. A few terpenes (e.g., 1,8-cineole, menthol, and menthone) are included in the list of Generally Recognized As Safe (GRAS) agents issued by the US Food and Drug Administration. Examples of terpenes suitable for the present disclosure may be selected from the group consisting of menthol, D-limonene, geraniol, nerolidol, and a mixture thereof.

Sulfoxide Permeation Enhancers

In certain embodiments, a PE mixture suitable for the instant disclosure comprises dimethylsulfoxide (DMSO), for enhancing the penetration of both hydrophilic and lipophilic APIs. Additional DMSO like PEs which may substitute for DMSO include similar, chemically related compounds such as Dimethylacetamide (DMAC), dimethylformamide (DMF), cyclic sulfoxides, decylmethyl sulfoxide, Dimethyl sulfoxide, and 2-Hydroxyundecyl methyl sulfoxide.

Glycol Permeation Enhancers

In certain embodiments, a PE mixture suitable for the instant disclosure comprises one or more glycol-based compounds such as a monoalkyl ether of diethylene glycol, preferably diethylene glycol monoethyl ether or diethylene glycol monomethyl ether or other dipropylene glycol, propylene glycol, 1,2-butylene glycol, etc. Currently, the Inactive Ingredients Database of the US Food and Drug Administration (FDA) lists diethylene glycol monoethyl ether (Transcutol) for topical (up to 49.9%) and transdermal (up to 5%) routes of administration. An important property of Transcutol is its capacity to dissolve a broad range of hydrophilic and lipophilic actives. Its ability to outperform PG and EtOH in solubilization power makes it a highly useful pharmaceutical excipient. With a negative log P of ˜0.5, Transcutol is considered as a polar protic solubilizer that demonstrates affinity and good miscibility with also hydrophobic groups. The ability of solvents having a negative log P to readily penetrate the stratum corneum contrasts with lipophilic actives (log P values of 2-3) more readily penetrating the stratum corneum than actives having negative log P values. Transcutol is compatible with most pharmaceutical excipients; soluble in common solvents like glycerin, ethanol, propylene glycol, and water; miscible with polar lipids like medium-chain triglycerides and polyethylene glycol based surfactants (polyoxylglycerides); but insoluble in non-polar mineral oil or dimethicone. Owing to its high solubility and miscibility with water, Transcutol may hydrate depending on the relative humidity conditions.

Emulsifying Agents

Producing a formulation for topical application to skin or mucosal surface can often require mixing an oil phase with an emulsifying agent. An emulsifying agent is a pharmaceutically acceptable surfactant, which may be a small molecule, oligomer or polymer. It may be nonionic, cationic or anionic. It may be of natural or synthetic origin.

Numerous emulsifying agents may be used in the instant disclosure. In certain embodiments, the emulsifying agent may comprise: sodium lauryl sulfate, or a non-ionic emulsifier (such as glyceryl stearate and/or PEG 100 stearate). Other representative emulsifiers include, but are not limited to, gelatin, casein, lecithin (phosphatides), gum acacia, cholesterol, tragacanth, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyoxyethylene stearates, colloidal silicon dioxide, sodium dodecylsulfate, carboxymethylcellulose calcium, carboxymethylcellulose sodium, methylcellulose, hydroxyethylcellulose, hydroxypropylcellulose, microcrystalline cellulose, and magnesium aluminum silicate. Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Other examples of surfactants include tyloxapol, poloxamers and polyxamines Poloxamers are water-soluble triblock copolymers composed of hydrophilic polyethylene oxide (PEO) and hydrophobic polypropylene oxide (PPO) blocks linked together. The amphiphilic nature of these block copolymers can be varied by controlling the length of the PEO and/or PPO block components (Ahmed et al., 2001). Several members of this poloxamer family of chemicals (such as poloxamer 188 and 407) are known to be biocompatible and non-toxic to mammalian cells and tissues, making them useful for biomedical applications. These surfactants are known to incorporate into or onto mammalian cell membranes, and thereby reduce protein adsorption and cell adhesion.

Still other emulsifiers include lecithin, dialkylesters of sodium sulfo succinic acid, such as Aerosol OT, which is a dioctyl ester of sodium sulfo succinic acid, available from American Cyanamid, Duponol P, which issodium lauryl sulfate, available from DuPont, Triton X-200, which is an alkyl aryl polyether sulfonate, available from Rohm and Haas, Tween® 20 and Tween® 80, which are polyoxyethylene sorbitan fatty acid esters, available from Croda, Inc.; Crodesta F-110, which is a mixture of sucrose stearate and sucrose distearate, available from Croda, Inc., Crodesta SL-40, which is available from Croda, Inc., and SA90HCO, which is Ci8H37-CH2(CON(CH3)CH2(CHOH)4CH2OH)2, decanoyl-N-methylglucamide; n-decyl-P-D-glucsopyranoside; n-decyl-P-D-maltopyranoside; n-dodecyl-P-D-glucopyranoside; n-dodecyl-P-D-maltoside; heptanoyl-N-methylglucamide; n-heptyl-P-D-glucopyranoside; n-heptyl-P-D-thioglucoside; n-hexyl-P-D-glucopyranoside; nonanoyl-N-methylglucamide; n-noyl-P-D-glucopyranoside; octanoyl-N-methylglucamide; n-octyl-P-D-glucopyranoside; octyl-P-D-thioglucopyranoside; and the like.

Another suitable surfactant is Vitamin E TPGS (α-tocopheryl polyethylene glycol succinate, also abbreviated as TPGS).

Many polymeric emulsifiers such as poloxamers and cellulosic excipients also act as gelling agents. Gels are semi-solid, three dimensional, polymeric matrices comprising small amounts of solid dispersed in relatively large amount of liquid, yet possessing more solid like character. Gels exhibit mechanical properties characteristic of the solid state, both the dispersed component and the dispersion medium extend themselves continuously throughout the whole system. Gels are often transparent or translucent semisolid formulations which are favored by patients due to their unobtrusiveness. Topical gel formulation provides a suitable delivery system for drugs because they are less greasy and provide better application property and stability in comparison to cream and ointments.

Other Ingredients

In certain embodiments, the compositions may further comprise one or more additives or combinations thereof, including but not limited to: wetting agents; texture enhancers; humidity regulators; pH regulators; osmotic pressure modifiers; UV-A and UV-B screening agents; and antioxidants. For example, antioxidants can be a-tocopherol, butylated hydroxyanisole or butylated hydroxytoluene, superoxide dismutase, ubiquinol, or certain metal-chelating agents. One skilled in this art will be able to select the optional compound(s) to be added to these compositions such that the advantageous properties intrinsically associated with the present disclosure are not, or are not substantially, adversely affected by the envisaged addition.

In addition, the compositions may further comprise one or more one or more additional active agents such as an antihistamine; a corticosteroid, a local anesthetic agent, a topical analgesic and an antibiotic. In various embodiments, the antihistamine may be diphenhydramine hydrochloride or chlorpheniramine maleate; the corticosteroid may be hydrocortisone, a hydrocortisone-21-monoester, (such as hydrocortisone-21-acetates, hydrocortisone-21-butyrate, hydrocortisone-21-propionate, hydrocortisone-21-valerate, etc., and a hydrocortisone-17,21-diester, (such as hydrocortisone-17,21-diacetate, hydrocortisone-17-acetate-21-butyrate, hydrocortisone-17,21-dibutyrate), dexamethasone, flumethasone, prednisolone, methylprednisolone, clobetasol propionate, betamethasone benzoate, betamethasone dipropionate, diflorasone diacetate, fluocinonide, mometasone furoate, or triamcinolone acetonide; the local anesthetic agent may be benzocaine, lidocaine, prilocaine and dibucaine; and the topical analgesic may be 1-menthol, d,1-camphor or capsaicin.

### Preferred Embodiments

The following are preferred embodiments of the present invention:
Embodiment 1. A topical Naproxen formulation, comprising
between about 0.5% wt % and about 25% wt % Naproxen;
between about 1.0 wt % and about 15.0 wt % of long chain monounsaturated fatty acids, long chain monounsaturated fatty alcohols, terpenes, or combinations thereof;
between 0 and about 5.0 wt % of a poloxamer;
between 0 and about 5.0 wt % of a pharmaceutically acceptable cellulosic excipient;
between 0 and about 5.0 wt % of α-tocopheryl polyethylene glycol succinate (“TPGS” or “Vitamin E TPGS”); and
a solvent mixture comprising ethanol, propylene glycol, 2-(2-Ethoxyethoxy)ethanol, and optionally dimethylsulfoxide (“DMSO”),
wherein the formulation comprises about 5.0 wt % or less water.
Embodiment 2. A topical Naproxen formulation according to embodiment 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between 0 and about 50 wt % 2-(2-Ethoxyethoxy)ethanol;
between about 1 wt % and about 5 wt % TPGS;
between 0 and about 50 wt % Poloxamer 188;
between 0 and about 5 wt % lidocaine;
between 0 and about 5 wt % cannabidiol;
between 0 and about 0.1 wt % vitamin D3; and
ethanol q.s. to 100 wt %.
Embodiment 3. A topical Naproxen formulation according to embodiment 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
between about 1 wt % and about 4 wt % TPGS;
between 0 and about 50 wt % Poloxamer 188;
between 0 and about 5 wt % lidocaine; and
ethanol q.s. to 100 wt %.

Embodiment 4. A topical Naproxen formulation according to embodiment 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
between about 1 wt % and about 5 wt % TPGS; and
ethanol q.s. to 100 wt %.
Embodiment 5. A topical Naproxen formulation according to embodiment 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol; and
ethanol q.s. to 100 wt %.
Embodiment 6. A topical Naproxen formulation according to one of embodiments 1-5, wherein the formulation comprises about 1.0 wt % or less water.
Embodiment 7. A topical Naproxen formulation according to one of embodiment 1-6, wherein the formulation is anhydrous.
Embodiment 8. A topical Naproxen formulation according to one of embodiments 1-7, wherein the formulation is transparent.
Embodiment 9. A topical Naproxen formulation according to one of embodiments 1-8, wherein the formulation has a refractive index between 1 and 2, measured using an Abbe refractometer in transmission mode at 20° C. at 589 nm.
Embodiment 10. A topical Naproxen formulation according to one of embodiments 1-9, wherein the formulation comprises between about at least 5 wt % ethanol, between about 10.0 and about 12 wt % propylene glycol, between about 15 wt % and about 45 wt % dimethylsulfoxide, and between about 10.0 and about 50 wt % 2-(2-Ethoxyethoxy)ethanol.
Embodiment 11. A topical Naproxen formulation according to one of embodiments 1-10, wherein the formulation comprises between about 5 wt % and about 15 wt % of a long chain monounsaturated fatty acid, a long chain monounsaturated alcohol, or mixtures thereof.
Embodiment 12. A topical Naproxen formulation according to one of embodiments 1-11, wherein the long chain monounsaturated fatty acid, a long chain monounsaturated alcohol, or mixtures thereof present in the formulation comprises or consists of oleic acid, oleyl alcohol, or a mixture thereof.
Embodiment 13. A topical Naproxen formulation according to one of embodiments 1-12, wherein the formulation comprises a poloxamer selected from the group consisting of poloxamer-101, -105, -105 benzoate, -108, -122, -123, -124, -181, -182, -182 dibenzoate, -183, -184, -185, -188, -212, -215, -217, -231, -234, -235, -237, -238, -282, -284, -288, -331, -333, -334, -335, -338, -401, -402, -403, and -407.
Embodiment 14. A topical Naproxen formulation according to embodiment 13, wherein the formulation comprises between about 2 wt % and about 5 wt % of poloxamer-188.
Embodiment 15. A topical Naproxen formulation according to one of embodiments 1-14, wherein the formulation comprises a pharmaceutically acceptable cellulosic excipient selected from the group consisting of hydroxypropylcellulose, hydroxypropyl methylcellulose, carboxymethylcellulose, methylycellulose, ethylcellulose, hydroxyethyl cellulose, hydroxyethylmethylcellulose and ethyl hydroxyethylcellulose.
Embodiment 16. A topical Naproxen formulation according to embodiment 15, wherein the formulation comprises between about 1 wt % and about 5 wt % of hydroxypropylcellulose.
Embodiment 17. A topical Naproxen formulation according to one of embodiments 1-16, wherein the formulation comprises between about 1 wt % and about 5 wt % of TPGS.
18. A topical Naproxen formulation according to embodiment 1, wherein the formulation is one of the following anhydrous formulations, wherein each value indicated is +/−10% of the indicated value, +/−1% of the indicated value, or is the indicated value:

| Ingredient | Wt % | Wt % | Wt % | Wt % |
|---|---|---|---|---|
| Oleic acid, oleyl alcohol, | 8.0 | 8.0 | 8.0 | 8.0 |
| or a combination thereof | | | | |
| DMSO | 20.0 | 20.0 | 20.0 | 20.0 |
| Ethanol | 33.0 | 32.0 | 31.0 | 30.0 |
| Naproxen | 1.0 | 2.0 | 3.0 | 4.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 8.0 | 8.0 | 8.0 | 8.0 |
| or a combination thereof | | | | |
| DMSO | 20.0 | 20.0 | 20.0 | 20.0 |
| Ethanol | 29.0 | 28.0 | 27.0 | 26.0 |
| Naproxen | 5.0 | 6.0 | 7.0 | 8.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 10.0 | 10.0 | 10.0 | 10.0 |
| or a combination thereof | | | | |
| DMSO | 20.0 | 20.0 | 20.0 | 20.0 |
| Ethanol | 23.0 | 22.0 | 21.0 | 20.0 |
| Naproxen | 9.0 | 10.0 | 11.0 | 12.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 20.0 | 20.0 | 20.0 | 20.0 |
| Ethanol | 17.0 | 16.0 | 15.0 | 14.0 |
| Naproxen | 13.0 | 14.0 | 15.0 | 16.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 13.0 | 13.0 | 13.0 |
| or a combination thereof | | | | |
| DMSO | 20.0 | 20.0 | 20.0 | 20.0 |
| Ethanol | 13.0 | 11.0 | 10.0 | 9.0 |
| Naproxen | 17.0 | 18.0 | 19.0 | 20.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 10.0 | 12.0 | 12.0 |
| or a combination thereof | | | | |
| DMSO | 20.0 | 20.0 | 20.0 | 20.0 |
| Ethanol | 15.0 | 15.0 | 9.0 | 20.0 |
| Naproxen | 15.0 | 15.0 | 15.0 | 10.0 |
| Lidocaine | 0 | 0 | 4.0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 2.0 | 2.0 | 0 |
| TOTAL | 100 | 100 | 100 | 100 |
| Ingredient | Wt % | Wt % | Wt % | Wt % |
| Oleic acid, oleyl alcohol, | 10.0 | 12.0 | 12.0 | 12.0 |
| or a combination thereof | | | | |
| DMSO | 20.0 | 20.0 | 20.0 | 20.0 |
| Ethanol | 20.0 | 12.0 | 21.0 | 17.0 |
| Naproxen | 10.0 | 10.0 | 5.0 | 5.0 |
| Lidocaine | 0 | 4.0 | 0 | 4.0 |
| Propylene Glycol | 10.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 2.0 | 2.0 | 2.0 |
| TOTAL | 100 | 100 | 100 | 100 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 45.0 | 45.0 | 0 | 0 |
| Ethanol | 18.0 | 13.0 | 18.0 | 9.0 |
| Naproxen | 10.0 | 15.0 | 10.0 | 15.0 |
| Lidocaine | 0 | 0 | 0 | 4.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 0 | 0 | 0 | 0 |
| TOTAL | 100 | 100 | 100 | 100 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 45.0 | 45.0 | 0 | 0 |
| Ethanol | 13.0 | 10.0 | 18.0 | 9.0 |
| Naproxen | 12.0 | 12.0 | 12.0 | 12.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 45.0 | 45.0 | 0 | 0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 45.0 | 45.0 | 0 | 0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 0 | 0 | 2.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 1.0 | 1.0 | 1.0 | 1.0 |
| TOTAL | 100 | 100 | 100 | 100 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 1.0 | 1.0 | 1.0 | 1.0 |
| TOTAL | 100 | 100 | 100 | 100 |

Embodiment 19. A topical Naproxen formulation according to embodiment 18, wherein the formulation is anhydrous.
Embodiment 20. A topical Naproxen formulation according to embodiment 18 or 19, wherein the formulation is transparent.
Embodiment 21. A topical Naproxen formulation according to one of embodiments 18-20, wherein the formulation has a refractive index between 1 and 2, measured using an Abbe refractometer in transmission mode at 20° C. at 589 nm.
Embodiment 22. A method of topically treating a pain episode at a location on the human body, comprising topically applying a topical Naproxen formulation according to one of embodiments 1-21 to the location.
Embodiment 23. A method according to embodiment 22, wherein the pain episode is an acute pain episode.
Embodiment 24. A method according to embodiment 22, wherein the pain episode is a chronic pain episode.
Embodiment 25. A method of topically treating pain of osteoarthritis of the knee(s), comprising topically applying a topical Naproxen formulation according to one of embodiments 1-21 to the knee(s).
Embodiment 26. A method according to one of embodiments 22-25, wherein the topical dose of Naproxen is about 80 mg, about 40 mg, about 30 mg, about 20 mg, or about 10 mg.

### Example 1. Formulations

The following is a procedure for preparing a naproxen formula of the invention.

Weigh naproxen, lidocaine, oleic acid, DMSO, DEGEE (Transcutol), propylene glycol, hydroxypropyl cellulose, vitamin E TPGS and anhydrous ethanol. At 20° C., combine DMSO and/or DEGEE while mixing to form solution A.

Combine propylene glycol and anhydrous ethanol, and add to solution A with mixing to form solution B. If formulation requires, add and dissolve Vitamin E TPGS in solution B before adding to solution A.

Dissolve naproxen or if formulation requires naproxen and lidocaine in solution B to form solution C.

Add oleic acid and/or oleyl alcohol to solution C to form solution D.

Slowly add the hydroxypropyl cellulose to solution D with vigorous mixing until polymer is completely solvated, the solution is homogenous and the proper viscosity between 500 and 3000 CPS is obtained to complete the formulation. The formulation is slightly yellow but transparent, has a refractive index between 1 and 2 with no opacity, no undissolved particles or co-crystals and has a viscosity between 500 and 3000 CPS (Centipoise).

One skilled in the art readily appreciates that the present disclosure is well adapted to carry out the objects and obtain the ends and advantages mentioned, as well as those inherent therein. The examples provided herein are representative of preferred embodiments, are exemplary, and are not intended as limitations on the scope of the disclosure.

It will be readily apparent to a person skilled in the art that varying substitutions and modifications may be made to the disclosure disclosed herein without departing from the scope and spirit of the disclosure.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

The disclosure illustratively described herein suitably may be practiced in the absence of any element or elements, limitation or limitations that is not specifically disclosed herein. Thus, for example, in each instance herein any of the terms “comprising”, “consisting essentially of” and “consisting of” may be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

Other embodiments are set forth within the following claims.

I claim:

1. A topical Naproxen formulation, comprising
between about 0.5% wt % and about 25% wt % Naproxen;
between about 1.0 wt % and about 15.0 wt % of long chain monounsaturated fatty acids, long chain monounsaturated fatty alcohols, terpenes, or combinations thereof;
between 0 and about 5.0 wt % of a poloxamer;
between 0 and about 5.0 wt % of a pharmaceutically acceptable cellulosic excipient;
between 0 and about 5.0 wt % of α-tocopheryl polyethylene glycol succinate (“TPGS” or “Vitamin E TPGS”); and
a solvent mixture comprising ethanol, propylene glycol, 2-(2-Ethoxyethoxy)ethanol, and optionally dimethylsulfoxide (“DMSO”),
wherein the formulation comprises about 5.0 wt % or less water.

2. A topical Naproxen formulation according to claim 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between 0 and about 50 wt % 2-(2-Ethoxyethoxy)ethanol;
between about 1 wt % and about 5 wt % TPGS;
between 0 and about 50 wt % Poloxamer 188;
between 0 and about 5 wt % lidocaine;
between 0 and about 5 wt % cannabidiol;
between 0 and about 0.1 wt % vitamin D3; and
ethanol q.s. to 100 wt %.

3. A topical Naproxen formulation according to claim 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
between about 1 wt % and about 4 wt % TPGS;
between 0 and about 50 wt % Poloxamer 188;
between 0 and about 5 wt % lidocaine; and
ethanol q.s. to 100 wt %.

4. A topical Naproxen formulation according to claim 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol;
between about 1 wt % and about 5 wt % TPGS; and
ethanol q.s. to 100 wt %.

5. A topical Naproxen formulation according to claim 1, wherein the formulation comprises:
between about 5% wt % and about 20% wt % Naproxen;
between about 5% wt % and about 15% wt % oleic acid, oleyl alcohol, or a combination thereof;
between about 15% wt % and about 45% wt % DMSO;
between about 5% wt % and about 15% wt % propylene glycol;
between about 1 wt % and about 5 wt % hydroxypropyl cellulose;
between about 10 wt % and about 30 wt % 2-(2-Ethoxyethoxy)ethanol; and
ethanol q.s. to 100 wt %.

6. A topical Naproxen formulation according to one of claims 1-5, wherein the formulation comprises about 1.0 wt % or less water.

7. A topical Naproxen formulation according to one of claim 1-6, wherein the formulation is anhydrous.

8. A topical Naproxen formulation according to one of claims 1-7, wherein the formulation is transparent.

9. A topical Naproxen formulation according to one of claims 1-8, wherein the formulation has a refractive index between 1 and 2, measured using an Abbe refractometer in transmission mode at 20° C. at 589 nm.

10. A topical Naproxen formulation according to one of claims 1-9, wherein the formulation comprises between about at least 5 wt % ethanol, between about 10.0 and about 12 wt % propylene glycol, between about 15 wt % and about 45 wt % dimethylsulfoxide, and between about 10.0 and about 50 wt % 2-(2-Ethoxyethoxy)ethanol.

11. A topical Naproxen formulation according to one of claims 1-10, wherein the formulation comprises between about 5 wt % and about 15 wt % of a long chain monounsaturated fatty acid, a long chain monounsaturated alcohol, or mixtures thereof.

12. A topical Naproxen formulation according to one of claims 1-11, wherein the long chain monounsaturated fatty acid, a long chain monounsaturated alcohol, or mixtures thereof present in the formulation comprises or consists of oleic acid, oleyl alcohol, or a mixture thereof.

13. A topical Naproxen formulation according to one of claims 1-12, wherein the formulation comprises a poloxamer selected from the group consisting of poloxamer-101, -105, -105 benzoate, -108, -122, -123, -124, -181, -182, -182 dibenzoate, -183, -184, -185, -188, -212, -215, -217, -231, -234, -235, -237, -238, -282, -284, -288, -331, -333, -334, -335, -338, -401, -402, -403, and -407.

14. A topical Naproxen formulation according to claim 13, wherein the formulation comprises between about 2 wt % and about 5 wt % of poloxamer-188.

15. A topical Naproxen formulation according to one of claims 1-14, wherein the formulation comprises a pharmaceutically acceptable cellulosic excipient selected from the group consisting of hydroxypropylcellulose, hydroxypropyl methylcellulose, carboxymethylcellulose, methylycellulose, ethylcellulose, hydroxyethyl cellulose, hydroxyethylmethylcellulose and ethyl hydroxyethylcellulose.

16. A topical Naproxen formulation according to claim 15, wherein the formulation comprises between about 1 wt % and about 5 wt % of hydroxypropylcellulose.

17. A topical Naproxen formulation according to one of claims 1-16, wherein the formulation comprises between about 1 wt % and about 5 wt % of TPGS.

18. A topical Naproxen formulation according to claim 1, wherein the formulation is one of the following anhydrous formulations 1-60, wherein each value indicated is +/−10% of the indicated value, +/−1% of the indicated value, or is the indicated value:

| | 1 | 2 | 3 | 4 |
|---|---|---|---|---|
| Ingredient: | Wt % | Wt % | Wt % | Wt % |
| Oleic acid, oleyl alcohol, | 8.0 | 8.0 | 8.0 | 8.0 |
| or a combination thereof | | | | |
| DMSO | 20.0 | 20.0 | 20.0 | 20.0 |
| Ethanol | 33.0 | 32.0 | 31.0 | 30.0 |
| Naproxen | 1.0 | 2.0 | 3.0 | 4.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| | 5 | 6 | 7 | 8 |
| Ingredient | Wt % | Wt % | Wt % | Wt % |
| Oleic acid, oleyl alcohol, | 8.0 | 8.0 | 8.0 | 8.0 |
| or a combination thereof | | | | |
| DMSO | 20.0 | 20.0 | 20.0 | 20.0 |
| Ethanol | 29.0 | 28.0 | 27.0 | 26.0 |
| Naproxen | 5.0 | 6.0 | 7.0 | 8.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| | 9 | 10 | 11 | 12 |
| Ingredient | Wt % | Wt % | Wt % | Wt % |
| Oleic acid, oleyl alcohol, | 10.0 | 10.0 | 10.0 | 10.0 |
| or a combination thereof | | | | |
| DMSO | 20.0 | 20.0 | 20.0 | 20.0 |
| Ethanol | 23.0 | 22.0 | 21.0 | 20.0 |
| Naproxen | 9.0 | 10.0 | 11.0 | 12.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 9 | 10 | 11 | 12 |
| TOTAL | 100 | 100 | 100 | 100 |
| | 13 | 14 | 15 | 16 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 20.0 | 20.0 | 20.0 | 20.0 |
| Ethanol | 17.0 | 16.0 | 15.0 | 14.0 |
| Naproxen | 13.0 | 14.0 | 15.0 | 16.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| | 17 | 18 | 19 | 20 |
| Ingredient | Wt % | Wt % | Wt % | Wt % |
| Oleic acid, oleyl alcohol, | 12.0 | 13.0 | 13.0 | 13.0 |
| or a combination thereof | | | | |
| DMSO | 20.0 | 20.0 | 20.0 | 20.0 |
| Ethanol | 13.0 | 11.0 | 10.0 | 9.0 |
| Naproxen | 17.0 | 18.0 | 19.0 | 20.0 |
| Lidocaine | 0 | 0 | 0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 0 | 0 | 0 |
| TOTAL | 100 | 100 | 100 | 100 |
| | 21 | 22 | 23 | 24 |
| Ingredient | Wt % | Wt % | Wt % | Wt % |
| Oleic acid, oleyl alcohol, | 12.0 | 10.0 | 12.0 | 12.0 |
| or a combination thereof | | | | |
| DMSO | 20.0 | 20.0 | 20.0 | 20.0 |
| Ethanol | 15.0 | 15.0 | 9.0 | 20.0 |
| Naproxen | 15.0 | 15.0 | 15.0 | 10.0 |
| Lidocaine | 0 | 0 | 4.0 | 0 |
| Propylene Glycol | 10.0 | 10.0 | 10.0 | 10.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 0 | 2.0 | 2.0 | 0 |
| TOTAL | 100 | 100 | 100 | 100 |
| | 25 | 26 | 27 | 28 |
| Ingredient | Wt % | Wt % | Wt % | Wt % |
| Oleic acid, oleyl alcohol, | 10.0 | 12.0 | 12.0 | 12.0 |
| or a combination thereof | | | | |
| DMSO | 20.0 | 20.0 | 20.0 | 20.0 |
| Ethanol | 20.0 | 12.0 | 21.0 | 17.0 |
| Naproxen | 10.0 | 10.0 | 5.0 | 5.0 |
| Lidocaine | 0 | 4.0 | 0 | 4.0 |
| Propylene Glycol | 10.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | | | | |
| Vitamin E TPGS | 25.0 | 25.0 | 25.0 | 25.0 |
| TOTAL | | | | |
| Vitamin E TPGS | 2.0 | 2.0 | 2.0 | 2.0 |
| TOTAL | 100 | 100 | 100 | 100 |
| | 29 | 30 | 31 | 32 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 45.0 | 45.0 | 0 | 0 |
| Ethanol | 18.0 | 13.0 | 18.0 | 9.0 |
| Naproxen | 10.0 | 15.0 | 10.0 | 15.0 |
| Lidocaine | 0 | 0 | 0 | 4.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 0 | 0 | 0 | 0 |
| TOTAL | 100 | 100 | 100 | 100 |
| | 33 | 34 | 35 | 36 |
| Ingredient | Wt % | Wt % | Wt % | Wt % |
| Oleic acid, oleyl alcohol, | 12.0 | 12.0 | 12.0 | 12.0 |
| or a combination thereof | | | | |
| DMSO | 45.0 | 45.0 | 0 | 0 |
| Ethanol | 13.0 | 10.0 | 18.0 | 9.0 |
| Naproxen | 12.0 | 12.0 | 12.0 | 12.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| | 37 | 38 | 39 | 40 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 45.0 | 45.0 | 0 | 0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| | 41 | 42 | 43 | 44 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 45.0 | 45.0 | 0 | 0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 0 | 0 | 45.0 | 45.0 |
| Vitamin E TPGS | 2.0 | 0 | 0 | 2.0 |
| TOTAL | 100 | 100 | 100 | 100 |
| | 45 | 46 | 47 | 48 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 0 | 0 | 2.0 |
| TOTAL | 100 | 100 | 100 | 100 |
| | 49 | 50 | 51 | 52 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 2.0 | 0 | 0 | 2.0 |
| TOTAL | 100 | 100 | 100 | 100 |
| | 53 | 54 | 55 | 56 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 3.0 | 3.0 | 3.0 | 3.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 1.0 | 1.0 | 1.0 | 1.0 |
| TOTAL | 100 | 100 | 100 | 100 |
| | 57 | 58 | 59 | 60 |
| Ingredient | Wt % | Wt % | Wt % | Wt % |
| Oleic acid, oleyl alcohol, | 8.0 | 10.0 | 8.0 | 10.0 |
| or a combination thereof | | | | |
| DMSO | 20.0 | 20.0 | 20.0 | 20.0 |
| Ethanol | 13.0 | 10.0 | 18.0 | 9.0 |
| Naproxen | 10.0 | 15.0 | 10.0 | 15.0 |
| Lidocaine | 2.0 | 2.0 | 2.0 | 2.0 |
| Propylene Glycol | 12.0 | 12.0 | 12.0 | 12.0 |
| Or CAPMUL PG | | | | |
| PG MonoLaurate | | | | |
| Hydroxypropyl Cellulose | 3.0 | 3.0 | 3.0 | 3.0 |
| 2-(2-Ethoxyethoxy)ethanol | 25.0 | 25.0 | 25.0 | 25.0 |
| Vitamin E TPGS | 1.0 | 1.0 | 1.0 | 1.0 |
| TOTAL | 100 | 100 | 100 | 100 |

19. A topical Naproxen formulation according to claim 18, wherein the formulation is anhydrous.

20. A topical Naproxen formulation according to claim 18 or 19, wherein the formulation is transparent.

21. A topical Naproxen formulation according to one of claims 18-20, wherein the formulation has a refractive index between 1 and 2, measured using an Abbe refractometer in transmission mode at 20° C. at 589 nm.

22. A method of topically treating a pain episode at a location on the human body, comprising topically applying a topical Naproxen formulation according to one of claims 1-21 to the location.

23. A method according to claim 22, wherein the pain episode is an acute pain episode.

24. A method according to claim 22, wherein the pain episode is a chronic pain episode.

25. A method of topically treating pain of osteoarthritis of the knee(s), comprising topically applying a topical Naproxen formulation according to one of claims 1-21 to the knee(s).

26. A method according to one of claims 22-25, wherein the topical dose of Naproxen is about 80 mg, about 40 mg, about 30 mg, about 20 mg, or about 10 mg.
